(19) United States
(12) Patent Application Publication
(10) Pub. No.: US 2005/0234475 A1
(43) Pub. Date: Oct. 20, 2005

(54) Transponder implanter

(76) Inventors: Christopher J. Cordes, Cannon Falls, MN (US); Richard W. Hughes, Cannon Falls, MN (US); Joseph V. Masin, Santa Barbara, CA (US)

Correspondence Address: PATTERSON, THUENTE, SKAAR & CHRISTENSEN, P.A., 4800 IDS CENTER, 80 SOUTH 8TH STREET, MINNEAPOLIS, MN, 55402-2100, US

(21) Appl. No.: 11/072,691

(22) Filed: Mar. 4, 2005

Related U.S. Application Data
(60) Provisional application No. 60/550,183, filed on Mar. 4, 2004.

(51) Int. Cl. A61B 17/00

(52) U.S. Cl. 606/117

(57) ABSTRACT

A transponder implanter for implanting an encapsulated transponder in living tissue, including a lancet, the lancet having a sharpened end and an interrupted cross sectional perimeter dimensioned to receive therein the encapsulated transponder. The lancet is connected to a lancet puller supported by a lancet barrel. A biasing member is interposed between the lancet puller and the lancet barrel and controlled by a release operably engagable to the lancet puller and capable of holding the lancet puller in a first position and resisting a biasing force operably applied to the lancet by the biasing member that tends to withdraw the lancet into the barrel. The release is also capable of releasing the puller such that the lancet withdraws into the barrel and a pushrod holds the encapsulated transponder substantially stationary relative to the barrel as the lancet is withdrawn.

### CLAIM TO PRIORITY

This application claims priority to U.S. Provisional application Ser. No. 60/550,183 filed Mar. 4, 2004 entitled “Transponder Implanter.” The entire contents of that application are incorporated herein by reference.

### FIELD OF THE INVENTION

The invention generally relates to the transcutaneous implantation of capsulized electronic transponders.

### BACKGROUND OF THE INVENTION

It is a commonly accepted practice to permanently identify animals by use of encapsulated electronic transponder implanted in the animal's body. Typically the electronic transponder capsule is implanted beneath the skin by the use of a hypodermic syringe and needle. This has been a generally accepted method of implanting transponder capsules in animal tissue for about the past twenty years. Quite often the hypodermic syringe used for implantation includes a push rod attached to the plunger that ejects the capsule from the end of the needle rather than using fluid pressure from the syringe barrel to eject the capsule. This method creates a number of difficulties that limit the efficacy of the implantation system and often creates unnecessary trauma to the animal receiving the implant.

A syringe implantable transponder generally includes an electronic capsule assembled inside the barrel of the needle or cannula. The entire syringe transponder assembly is typically delivered to the user in a sealed sterilizeable package. The syringe needle typically is typically a 12-gauge cannula. The encapsulated electronic transponder is generally cylindrical with rounded ends and about 2 mm in diameter. The manufacturing tolerances achievable in manufacturing transponders are such that the difference between the inside diameter of the cannula and the outside diameter of the transponder is often enough to prevent a snug fit within the cannula to keep the transponder retained in the cannula until the desired time of implantation.

Various efforts have been made to retain the capsule inside the needle barrel. These include adding an adhesive or a gel to fill the space between the surface of the capsule and the inside surface of the cannula. Another approach is to place a resilient plastic cap over the end of the glass capsule that contains the transponder to fill the space between the glass capsule and the cannula. Yet another approach is to impress an indentation into the needle a short distance from where the capsule is inserted to constrict inside of the needle to retain the capsule. The transponder capsule is typically formed from thin glass. Therefore, the mechanical constriction approach has the obvious risk of fracturing the glass capsule thus destroying the sealed nature of the glass capsule. Exposing the electronics within the transponder to moisture after implantation leads to rapid deterioration of the electronics.

The addition of an adhesive or gel also creates a risk of capsule breakage by increasing the capsule's resistance to ejection. The increased resistance to ejection can require application of sufficient force to the ejecting rod to fracture the sealed glass capsule. Again this leads to rapid deterioration of the electronics in the transponder. The use of an adhesive gel or plastic cap also introduces additional foreign material into the living tissue of the animal increasing the likelihood of immune response, inflammation and consequent expression of the encapsulated transponder.

A hypodermic syringe needle is a closed tubular structure that prevents visual inspection of the transponder capsule within. Typically the hypodermic syringe needle is fully assembled including a push rod traveling within the syringe to expel the encapsulated transponder. The closed metallic tube structure also restricts or eliminates the possibility of electronically verifying the presence of the capsule or testing its circuitry prior to implantation. The closed metallic nature of the cannula severely interferes with electromagnetic fields that are necessary to communicate with the transponder.

Thus, many concerns arise each time an attempt is made to place a syringe implantable transponder within an animal body. First, with regard to the transponder itself, there is no assurance that there is a capsule within the needle. Second, there is the possibility that multiple capsules may be within the needle. Additionally there can be doubt as to whether there is a push rod present. There is no way to examine the capsule to determine whether it may be physically damaged. Finally, there is no way to test the electronic functionality of the capsule prior to implantation.

Other concerns arise regarding proper localization of the transponder in the animal. It is important that the capsule be placed in a predetermined location within the animal's body. Further it is important that the placement be accomplished without damaging the encapsulated transponder. Finally it is important that the procedure cause minimal trauma to the animal tissue.

When inserting a transponder it is possible to visually gauge the depth to which the transponder is inserted into tissue by inserting the needle to a predetermined depth. However, during ejection of the capsule from the needle, other factors come into play, which may alter the ultimate location of the transponder inserted from that which would be ideal.

Variables that may affect the placement of the encapsulated transponder include movement of the animal. If the needle is inserted to a certain depth and the capsule is then pushed out of the needle past the point where the needle's tip has made a tract in the tissue substantial force is required to advance the capsule. This increases the risk of fracturing the glass capsule surrounding the transponder. In some species a desired location of implantation may be subcutaneous tissue. In other species the desired location may be deeper in firm muscle or ligament tissue. It is of course possible that a needle may be inserted to the point where it rests against a bone. This creates a substantial risk that the application of force to the plunger will force the capsule against the bone and break the capsule.

A person performing an implantation may choose to try to avoid the risk of capsule breakage and increased trauma to the animal by simultaneous withdrawing the needle as the plunger is pushed, to eject the capsule from the end of the needle. To do this effectively requires a coordinated motion that is difficult to master. The partial withdrawal of the needle while the capsule is being ejected creates an additional risk that the needle will be removed too far before full ejection of the capsule occurs. If the needle is withdrawn too far the capsule will not be implanted at its desired location. This may leave the capsule too near the opening of the incision or even outside the skin trapped in animal's fur or hair. Very often the person implanting the capsule may think the procedure has been successfully accomplished while the capsule is not located within the incision or the capsule is so near the surface of the skin that the capsule is later expelled.

Another concern raised by the syringe placement of encapsulated transponders is that of the trauma caused by a very sharp needle moving within the animal tissue because of movement of implanting individual's hand and/or movements of the animal. Trauma of this sort may sever additional blood vessels causing increased blood flow out of the wound, which may tend to push the capsule out of the incision. Increased trauma to the implantation area will also tend to created inflammation and potentially longer healing time and a greater opportunity for the capsule to migrate out of the intended location to other parts of the body or to the outside of the skin.

In addition, during the time that the capsule is exiting the tip of the needle it is very susceptible to breakage due to the lateral forces caused by movements of the needle or the animal. Very small lateral forces may fracture a capsule. Thus, it is important to minimize exit time during which the capsule is transitioning from within the needle to outside the needle implanted within the animal.

Thus it would be valuable to those who implant encapsulated transponders to have a way to implant the transponders predictably in a desired location without damage to the capsule. In addition it would be beneficial to facilitate verification that the capsule is present within the implanting device and that the capsule is undamaged and fully functional prior to the implantation.

### SUMMARY OF THE INVENTION

The invention resolves many of the above problems by providing a transponder lancet implanter to implant encapsulated transponders. The transponder lancet is a resilient channel structure having a cross section the perimeter of which is interrupted such as C-shaped, U-shaped or V-shaped and a sharpened end. For the purposes of this application, the term C-shaped should be understood to encompass all of these shapes and other shapes having an interrupted cross sectional perimeter and the term interrupted cross sectional perimeter means any cross section shape that is not a closed curve. The C-shaped channel structure resiliently holds a transponder capsule so that the transponder capsule can be maintained in position in the lancet prior to implantation by the spring tension of the upwardly rolled sides of the C-shaped channel. Desirably the dimensions and resiliency of the channel are such that they apply enough force to maintain the position of the capsule without danger of fracturing the fragile glass capsule structure. The lancet structure is not a closed tube; it has a slit like opening along its entire length. The opening allows for visual inspection of the encapsulated transponder up until the moment of implantation. This open structure also provides for relatively uninhibited electromagnetic field “visibility” to the capsule electronics. This allows for testing and/or programming of the encapsulated transponder until implantation. In addition, this embodiment of the invention eliminates the need for the use of any adhesive gels or liquids, which may introduce additional foreign matter into the animal tissue upon implantation.

The present invention holds the encapsulated transponder in position relative to the lancet during the creation of the lancet incision and during transcutaneous advancement of the lancet into the animal tissue. Both the lancet and the transponder are held in position relative to a holder gripped by the person performing the insertion. The lancet is secured against a stop during the insertion of the lancet into the tissue. Thus force applied to insert the lancet is transmitted through the lancet to advance it into the tissue while the transponder is held stationary relative to the lancet. After the desired depth of in implantation is achieved, the stop is released and the lancet is retracted relative to the transponder leaving the transponder implanted at a desired depth beneath the skin.

A buffer/gauge is positioned above the transponder within the transponder lancet so that the buffer/gauge is in contact with the transponder itself.

The transponder lancet resiliently grips the transponder in the lancet until the lancet is withdrawn from around the transponder leaving the transponder properly positioned beneath the skin of an animal. The transponder lancet has a sharp first end that facilitates insertion into the tissue of an animal and a second end coupled to a lancet holder. As the sharp first end enters the skin of an animal it creates a C-shaped incision as opposed to a puncture in the skin. Thus, the structure of the transponder lancet creates a retained operculum or flap of skin that covers the wound after withdrawal of the lancet. The operculum facilitates wound healing of the skin after insertion of the device.

During the implantation process, after the lancet is inserted, the transponder is not pushed or injected into the animal, instead the transponder is held in place at a desired location while the lancet is withdrawn from around the transponder, thereby subjecting the transponder to less stress and reducing the risk of damage to the transponder.

One embodiment of the invention is intended primarily for use in a veterinary office setting. This embodiment of the invention includes a lancet holder with a spring actuated withdrawal mechanism. The lancet is loaded into the lancet holder whereupon a portion of the lancet holder is advanced to engage a pawl to the rear end of a buffer/gauge. The buffer/gauge is then held stationary relative to the lancet holder while the lancet is inserted into the tissue of an animal. Once the lancet is inserted to the desired depth within the animal tissue, activation of a trigger releases the lancet so that the lancet is withdrawn briskly into the lancet holder while the buffer/gauge is held stationary relative to the lancet holder, thus the buffer/gauge holds the transponder capsule in a substantially fixed location while the lancet is withdrawn.

Another embodiment of the invention is intended for use in a more rugged location, such as a livestock barn or with livestock animals in a field environment. The invention generally includes a lancet fixed to a lancet holder that has one or two finger grips to facilitate retraction of the lancet. The lancet holder is positioned in a lancet housing that includes a tubular receiving structure and a palm rest formed to fit conveniently in the palm of a user's hand.

The lancet holder is inserted into the lancet retractor where it comes against an initial stop. The initial stop holds the lancet so that it is exposed beyond the end of the lancet retractor by a measured distance as required to place the lancet below the skin at a desired depth. The lancet holder may then be moved beyond the stop, for example, by twisting relative to the lancet retractor. The lancet holder may then be pulled back relative to the lancet retractor whereupon a rod located within the lancet retractor stops the motion of a buffer/gauge within the lancet allowing the lancet to be withdrawn while the transponder is held in place by the buffer/gauge at a desired depth.

The rod or positioner is desirably a static steel pin firmly imbedded within the lancet retractor.

The buffer/gauge is positioned so that it falls between the positioner and the transponder. The buffer/gauge prevents damage to the transponder during retraction of the lancet. Desirably, the buffer/gauge is formed from a brightly colored plastic to distinguish it from the metal coloring of the lancet and the coloring of the transponder. Thus the buffer/gauge provides an accurate visual reference of the depth of the implantation of the transponder.

In this embodiment the lancet holder is positioned in the lancet retractor and advanced to the first stop. Then, an operator uses the lancet to create an incision directly into the skin and underlying tissues of an animal. The stop prevents retraction of the lancet during insertion. The positioner secures the location of the transponder within the lancet. Once the lancet is fully inserted, the operator rotates the lancet holder or otherwise releases it from the stop. The operator then holds the lancet retractor in place with his palm while pulling the lancet holder toward the proximal end of the lancet retractor, thus retracting the lancet while leaving the transponder is a desired location. The lancet and lancet holder are then withdrawn from the animal.

### BRIEF DESCRIPTION OF THE DRAWINGS

FIG. 1 is an exploded perspective view of a transponder implanter in accordance with the present invention.

FIG. 2 is a plan view of the transponder implanter shown in FIG. 1.

FIG. 3 is a side elevational view of the transponder implanter shown in FIG. 1.

FIG. 4 is an end elevational view of the transponder implanter shown in FIG. 1.

FIG. 5 is a sectional view of the transponder implanter of the present invention taken along section line 5-5 of figure.

FIG. 6 is a perspective view of a lancet in accordance with the present invention.

FIG. 7 is a side elevational view of the lancet as shown in FIG. 6.

FIG. 8a is sectional view of the lancet taken along section line 8a-8a of FIG. 7.

FIG. 8b is sectional view of the lancet taken along section line 8b-8b of FIG. 7.

FIG. 9 is a perspective view of an encapsulated transponder as utilized with the present invention.

FIG. 10 is a perspective view of a transponder implanter in accordance with another embodiment of the present invention.

FIG. 11 is a perspective view of the transponder implanter of FIG. 10 with a lancet holder partially retracted.

FIG. 12 is a perspective view of a lancet holder of the present invention.

FIG. 13 is a perspective view of a lancet holder in accordance with the present invention.

FIG. 14 is a perspective view of a lancet holder in accordance with the present invention.

### DETAILED DESCRIPTION OF THE INVENTION

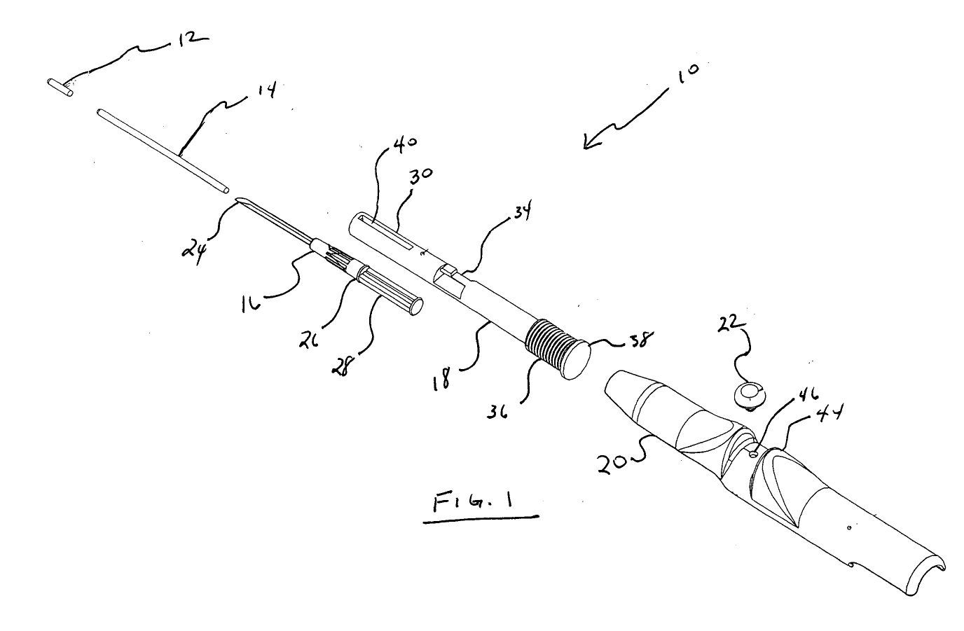

Referring to the FIG. 1, an embodiment of the transponder implanter 10 for implanting encapsulated transponder 12 generally includes pushrod 14, transponder lancet 16, lancet puller 18, lancet barrel 20 and push button 22. Transponder lancet 16 includes a resilient channel structure having a cross section enclosing less than the full perimeter of the transponsder 12 and a sharpened end 24. The cross section may be C-shaped, U-shaped or V-shaped for example. Transponder lancet 16 further includes support collar 26 and breech 28.

Pushrod 14 and encapsulated transponder 12 are dimensioned to fit snugly within transponder lancet 16. Transponder lancet 16 is resilient so that the C-shaped cross section securely grips and retains encapsulated transponder 12 and pushrod 14 within while allowing movement of encapsulated transponder 12 and pushrod 14 without the application of excessive force. Transponder implanter 10 is formed from materials that will withstand sterilization.

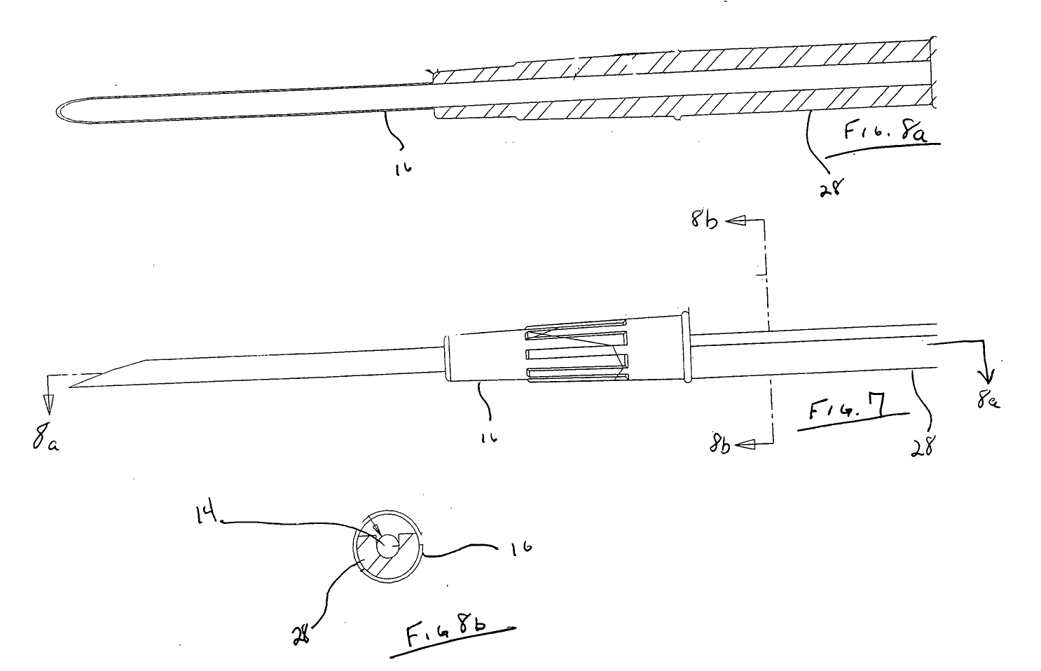

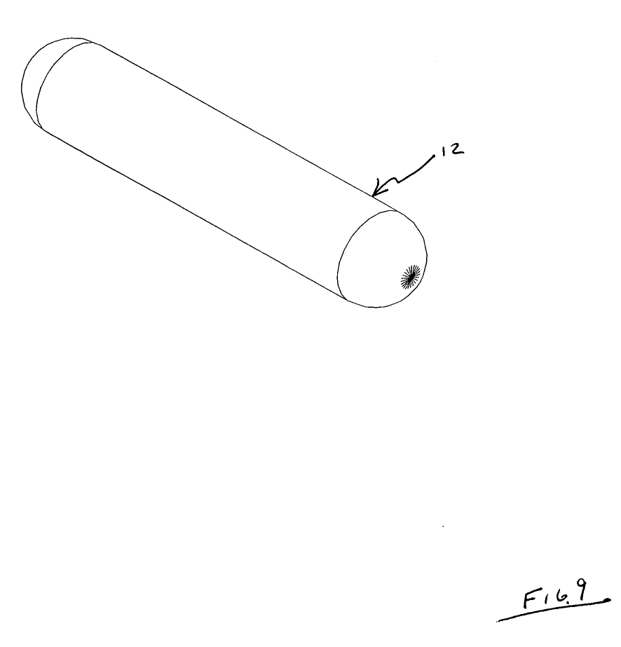

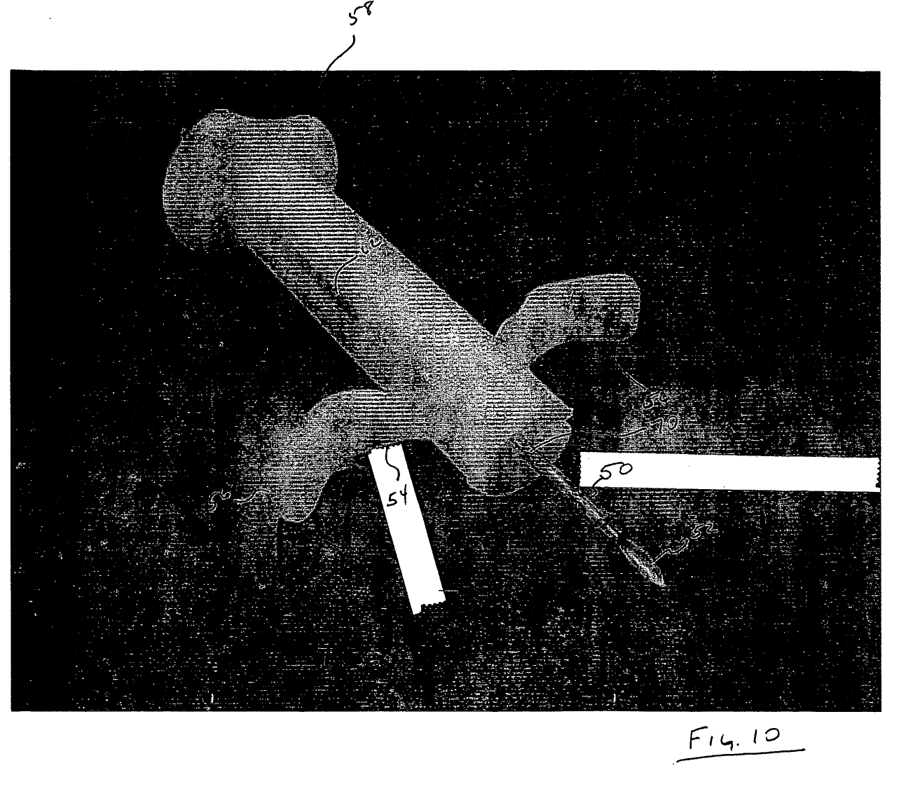

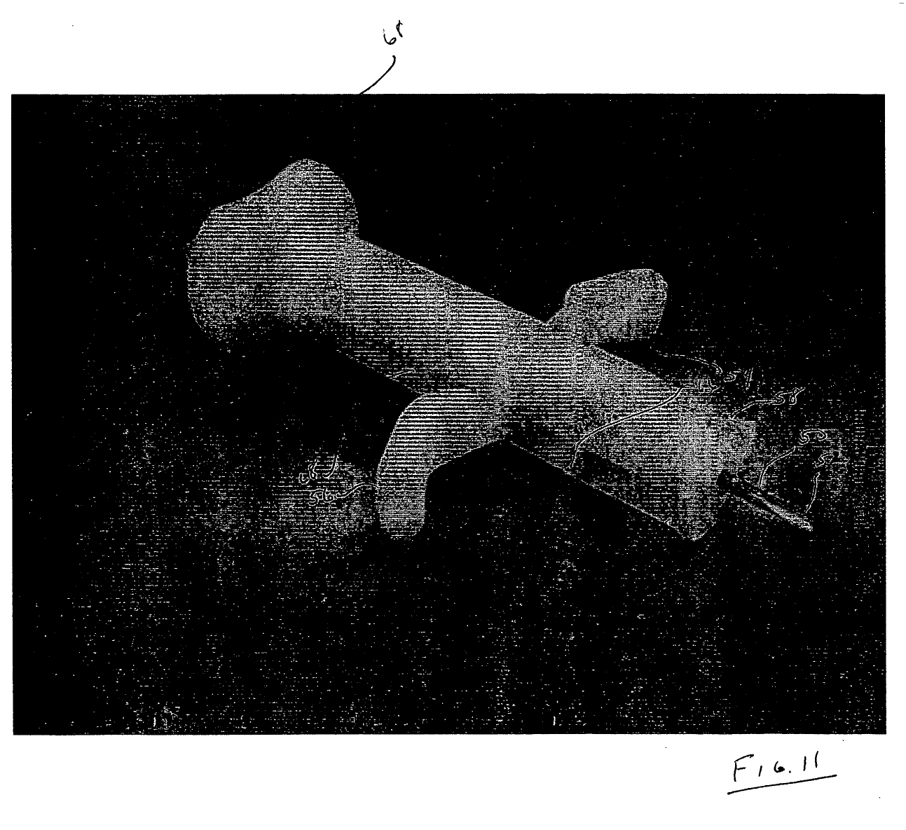

Lancet puller 18, as depicted in FIG. 5, is desirably formed of a molded polymer material and includes sleeve 30, collar grip 32, releasable catch 34, spring 36 and spring retainer 38. Slot 40 pierces sleeve 30.

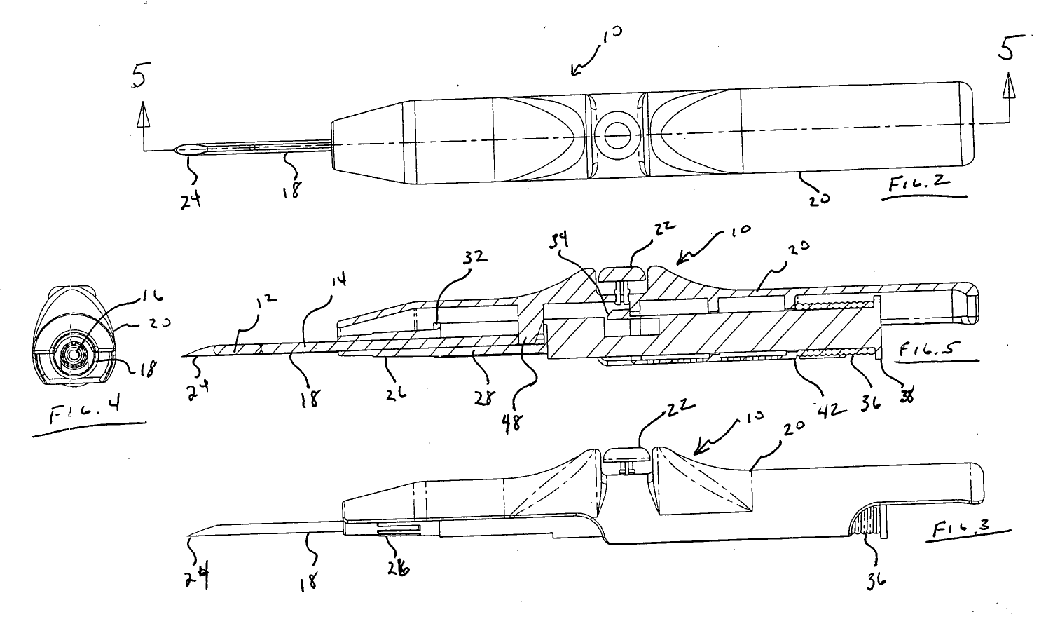

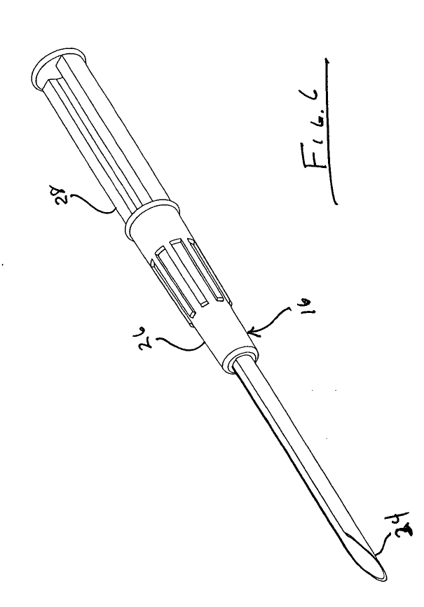

Referring to FIGS. 2-5, lancet barrel 20 is adapted to receive lancet puller 18 therein. Lancet barrel 20 is desirably formed of a rigid polymer material. Lancet barrel 20 generally includes spring boss 42, handgrip 44 and pushbutton receiver 46. Spring 36 rests against spring boss 42 on lancet barrel 20 and against spring retainer 38 on lancet puller 18, so that spring 36 biases lancet puller 18 in a direction away from transponder lancet 16. Push button 22 fits into push button receiver 46 and impinges upon releasable catch 34 so that when push button 22 is actuated, releasable catch 34 releases lancet puller 18 so that the bias of spring 36 pulls lancet puller 18 in a direction away from transponder lancet 16. Lancet 16 is secured to lancet puller 18 and is thus withdrawn rapidly into lancet barrel 20. Lancet barrel 20 further includes pawl 48. Pawl 48 is located so as to pass through slot 40. Pawl 48 engages the end of pushrod 14 holding pushrod 14 stationary while transponder lancet 16 is withdrawn. Thus holding transponder 12 stationary relative to the animal being implanted and leaving transponder 12 in place after lancet 16 is withdrawn.

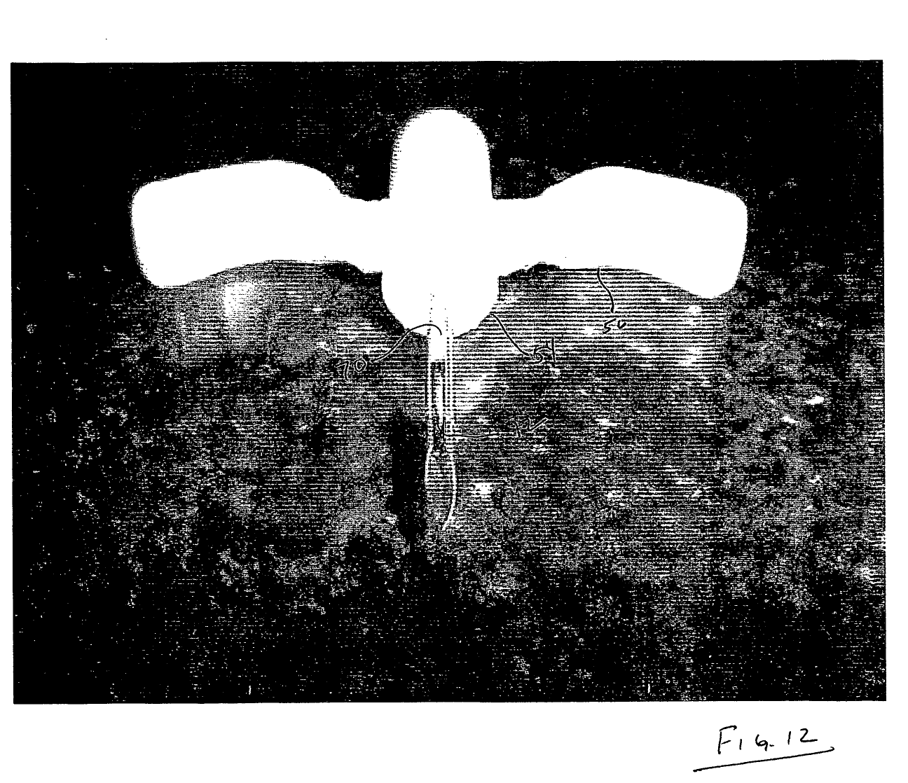

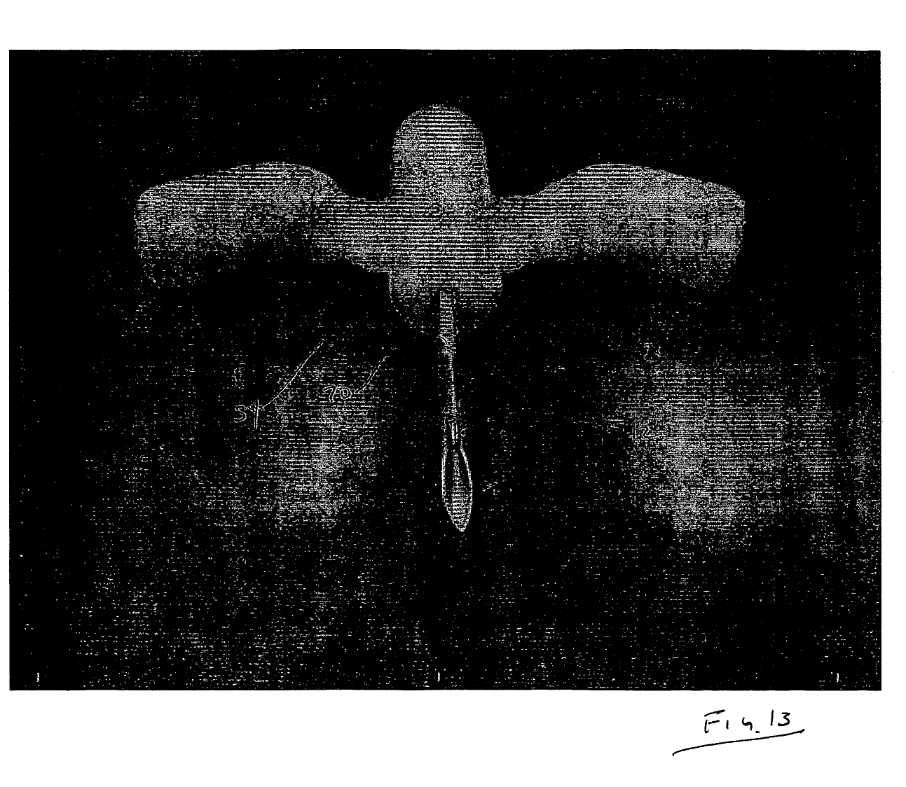

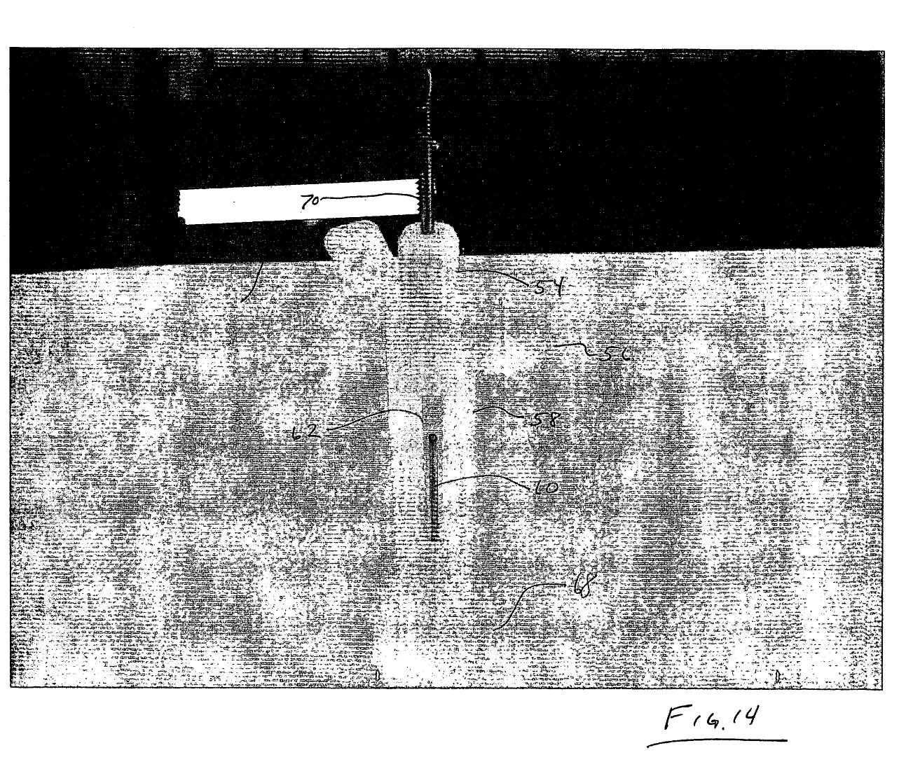

Another embodiment of the invention, intended for use in more rugged environments, such as that encountered when implanting live stock animals in a field or barn is depicted in FIG. 10. This embodiment also includes transponder lancet 50 which is structured similarly to transponder lancet 16 above. Transponder lancet 50 again includes a C-shaped, U-shaped or V-shaped channel having a sharpened end 52. Lancet holder 54 supports lancet 50 and includes two finger grips 56. This embodiment of the invention generally includes transponder lancet 50, lancet holder 54, lancet housing 58 and positioner 60. Desirably lancet holder 54 is dimensioned to be received within lancet housing 58 in a telescoping fashion. Lancet housing 58 includes interrupted slits 62. Interrupted slits 62 each included an initial straight portion 64, stop 66 and second straight portion 68. Desirably interrupted slits 62 are oriented about 180° apart in lancet housing 58. Lancet housing 58 also supports positioner 60. Positioner 60 is desirably a rigid pin or rod firmly imbedded within lancet housing 58.

Lancet housing 58 also includes palm rest 68. Palm rest 68 is dimensioned and shaped to rest comfortably in the palm of an operator's hand. Lancet holder 54 is insertable into interrupted slits 62 in lancet housing 58. Initially, finger grips 56 are fit into straight portion 64 of interrupted slits 62. Lancet holder 54 is inserted straight inwardly until it rests against stop 66.

When it is desired to withdraw transponder lancet 50 from around implanted encapsulated transponder 12, lancet holder 54 is rotated so that finger grips 56 no longer impinge upon stop 66. Lancet holder 54 may then be withdrawn along second straight portion 67 of interrupted slits 62. This is conveniently done by pulling back on finger grips 56 with two fingers while palm rest 68 rests in the palm of the hand. When lancet holder 54 is withdrawn, positioner 60 holds buffer/gauge 70 in position relative to the animal tissue which in turn holds encapsulated transponder 12 at a desired depth relative to the animal's skin as transponder lancet 50 is withdrawn from around encapsulated transponder 12, thus leaving encapsulated transponder 12 in a desired position.

Buffer/gauge 70 fits snugly within transponder lancet 50 and is desirably made of a brightly colored plastic material to visually distinguish it from the lancet and the transponder. Thus buffer/gauge 70 provides a convenient and accurate visual reference indicating the depth of implantation.

In operation of this embodiment, encapsulated transponder 12 and buffer/gauge 70 are inserted into transponder lancet 50. Encapsulated transponder 12 is located closer to sharpened end 52 than is buffer/gauge 70. Lancet holder 54 is then inserted into straight portion 64 of interrupted slits 62 on lancet housing 58 until finger grips 56 rest against stop 66. Transponder lancet 54 is then inserted into animal tissue to a desired depth by visual reference to buffer/gauge 70. When a desired depth is achieved, lancet holder 54 is rotated relative to lancet housing 58, so that finger grips 56 no longer rest against stop 66. The operator grasps finger grips 56 while holding palm rest 68 against the palm of the operator's hand. Finger grips 56 are then withdrawn toward palm rest 68, thereby retracting lancet holder 54 into lancet housing 58. Positioner 60 remains in position holding buffer/gauge 70 and consequently encapsulated transponder 12 in location. Transponder lancet 50 is withdrawn, leaving encapsulated transponder in a desired location. Transponder lancet 50 is then withdrawn completely from the animal tissue allowing the healing process to begin so that encapsulated transponder 12 is permanently implanted into animal tissue.

The present invention may be embodied in other specific forms without departing from the central attributes thereof, therefore, the illustrated embodiment should be considered in all respects as illustrative and not restrictive, reference being made to the appended claims rather than the foregoing description to indicate the scope of the invention.

1. A transponder implanter for implanting an encapsulated transponder in living tissue, comprising
a lancet, the lancet comprising a sharpened end and having an interrupted cross sectional perimeter and being dimensioned to receive therein the encapsulated transponder and to grip the transponder;
a lancet puller capable of operably, releasably engaging the lancet;
a lancet barrel supporting the lancet puller,
a biasing member interposed between the lancet puller and the lancet barrel;
a release operably engagable to the lancet puller capable of holding the lancet puller in a first position and resisting a biasing force operably applied to the lancet by the biasing member that tends to withdraw the lancet into the barrel and also capable of operably releasing the puller such that the lancet withdraws into the barrel under the influence of the biasing force; and
a pushrod operably engageable to the barrel capable of holding the encapsulated transponder substantially stationary relative to the barrel as the lancet is withdrawn.

2. The transponder implanter as claimed in claim 1, in which the barrel further comprises a pawl and the lancet puller defines a slot therethrough and the pawl passes through the slot to engage the pushrod.

3. The transponder implanter as claimed in claim 1, in which the lancet further comprises a support collar, the support collar being engageable to a collar grip on the lancet puller whereby the lancet puller operably engages the lancet and withdraws the lancet into the barrel.

4. The transponder implanter as claimed in claim 2, in which the lancet further comprises a breech in which the breech is dimensioned to receive the pushrod and the pawl therein.

5. The transponder implanter as claimed in claim 1, in which the release comprises a releasable catch located on the lancet puller and a finger actuated member on the barrel.

6. A method of implanting encapsulated transponders into animal tissue, the method comprising the steps of:
inserting the encapsulated transponder into a lancet comprising a sharpened end and having an interrupted cross sectional perimeter and being dimensioned to receive therein the capsulized transponder and to grip the transponder;
engaging the lancet to a lancet puller capable of operably, releasably engaging the lancet, the lancet being supported by a lancet barrel, a biasing member being interposed between the lancet puller and the barrel;
cocking the lancet puller in the barrel;
inserting the lancet into the animal tissue;
releasing the cocked lancet puller so that the lancet is withdrawn from the animal tissue and into the barrel while the encapsulated transponder is held substantially stationary relative to the barrel and within the animal tissue.

7. The method as claimed in claim 6 further comprising the step of engaging a pawl on the barrel with a pushrod in the lancet via a slot in the lancet puller.

8. The method as claimed in claim 6 further comprising the step of engaging a support collar on the lancet to a collar grip on the lancet puller.

9. The method as claimed in claim 7, further comprising the step of inserting a pushrod into a breech of the lancet in which the breech is dimensioned to receive the pushrod and the pawl therein and in which the pushrod contacts the transponder.

10. The method as claimed in claim 6 further comprising the step of actuating a releasable catch located on the lancet puller via a finger actuated member on the barrel.

11. A transponder implanter for implanting an encapsulated transponder in living tissue, comprising
a lancet, the lancet comprising a sharpened end and having an interrupted cross sectional perimeter and being dimensioned to receive therein the encapsulated transponder and to grip the transponder;
means for pulling the lancet capable of operably engaging the lancet;
means for supporting the means for pulling,
means for biasing the lancet interposed between the means for pulling and the means for supporting; the means for biasing being biased toward withdrawing the lancet into the means for supporting;
means for operably engaging the means for pulling capable of holding the means for pulling in a first position and resisting a biasing force operably applied to the lancet by the means for biasing and also capable of operably releasing the means for pulling such that the lancet withdraws into the means for supporting under the influence of the biasing force; and
means for holding the transponder substantially stationary relative to the means for supporting as the lancet is withdrawn

12. The transponder implanter as claimed in claim 11, in which the means for supporting further comprises a pawl and the lancet puller defines a slot therethrough and the pawl passes through the slot to engage the means for holding the transponder substantially stationary.

13. The transponder implanter as claimed in claim 11, in which the lancet further comprises a support collar, the support collar being engageable to a collar grip on the means for pulling whereby the means for pulling operably engages the lancet and withdraws the lancet into the means for supporting.

14. The transponder implanter as claimed in claim 12, in which the lancet further comprises a breech in which the breech is dimensioned to receive the means for holding sub means for holding the transponder substantially stationary and the pawl therein.

15. The transponder implanter as claimed in claim 11, in which the means for operably engaging the means for pulling comprises a releasable catch located on the means for pulling and a finger actuated member on the means for supporting.
